(19) United States
(12) Patent Application Publication
(10) Pub. No.: US 2024/0415910 A1
(43) Pub. Date: Dec. 19, 2024

(54) Toxicity-Removed and Medicinal Effect-Retained Fuzi Decoction Granule and Preparation Method Thereof

(71) Applicant: Zhejiang Chinese Medical University, Hangzhou City (CN)

(72) Inventors: Letian SHAN, Hangzhou City (CN); Li ZHOU, Hangzhou City (CN)

(21) Appl. No.: 18/744,424

(22) Filed: Jun. 14, 2024

(30) Foreign Application Priority Data
Jun. 15, 2023 (CN) 202310714521.2

(51) Int. Cl. A61K 36/076; A61K 9/16; A61K 31/439; A61K 36/258; A61K 36/284; A61K 36/54; A61K 36/65; A61K 36/714

(52) U.S. Cl. CPC A61K 36/076; A61K 9/1652; A61K 9/1694; A61K 31/439; A61K 36/258; A61K 36/284; A61K 36/54; A61K 36/65; A61K 36/714; A61K 2236/331; A61K 2236/51; A61K 2236/53

(57) ABSTRACT

A toxicity-removed and medicinal effect-retained Fuzi decoction granule and a preparation method thereof are provided, belonging to the technical field of traditional Chinese medicine preparation. The preparation method of the toxicity-removed and medicinal effect-retained Fuzi decoction granule includes the following steps: subjecting Radix Aconiti Lateralis Preparata to immersion and decoction in advance for 60 min to 120 min, and then mixing with Poria, Radix Ginseng, bran-fried Rhizoma Atractylodis Macrocephalae, Radix Paeoniae Alba, Radix Paeoniae Rubra, and Ramulus Cinnamomi to allow immersion for 30 min to 60 min; and subjecting a filtrate obtained by filtration to concentration and granulation in sequence to obtain the toxicity-removed and medicinal effect-retained Fuzi decoction granule. The Fuzi decoction granule prepared by the preparation method can remove inherent toxicity and retain a medicinal effect on osteoarthritis.

### CROSS REFERENCE TO RELATED APPLICATION

This patent application claims the benefit and priority of Chinese Patent Application No. 202310714521.2, filed with the China National Intellectual Property Administration on Jun. 15, 2023, the disclosure of which is incorporated by reference herein in its entirety as part of the present application.

### TECHNICAL FIELD

The present disclosure belongs to the technical field of traditional Chinese medicine preparation, and in particular relates to a toxicity-removed and medicinal effect-retained Fuzi decoction granule and a preparation method thereof.

### BACKGROUND

Knee osteoarthritis (kOA) is a joint degenerative disease with the highest clinical incidence and the second largest cause of disability, seriously affecting people's quality of life. The KOA occurs usually in middle-aged and elderly people and is related to genetic and physical factors; trauma, inflammation, accumulated strain or congenital disease can also cause kOA. Cross-sectional studies and meta-analyses show that the overall prevalence of kOA in China is about 18%, and the incidence of kOA has been increasing annually with the aging of population and the intensification of obesity in China. KOA can cause joint pain, deformity, and mobility dysfunction, thereby increasing the incidence of cardiovascular events and all-cause mortality, where the all-cause mortality nearly doubles with kOA.

Medication is an important part of kOA treatment. The “Consensus of Traditional Chinese Medicine Experts on the Diagnosis and Treatment of Knee Osteoarthritis” states that the main TCM syndrome type of kOA is cold-dampness arthralgia syndrome, commonly known as “old cold legs” in Chinese. Fuzi decoction granule (also called aconite decoction granule) comes from “Treatise on Febrile Diseases” and is mainly used to treat patients with Yang deficiency and internal invasion of cold dampness as well as joint pain in the body. Fuzi decoction granule serves as a classic prescription for warming Yang and dispersing cold, and is suitable for the treatment of kOA. In the prescription, Radix Aconiti Lateralis Preparata replenishes fire and revitalizes primordial yang, warms the meridians and dispels cold; Radix Ginseng (dried root of Panax ginseng C. A. Mey.) greatly replenishes vitality and regenerates qi and blood; Poria strengthens the spleen and exudes dampness, and helps Radix Ginseng and Radix Aconiti Lateralis Preparata (lateral root of Aconitum carmichaeli Debx.) to invigorate qi and warm yang; Rhizoma Atractylodis Macrocephalae invigorates spleen to dispel dampness, dehumidification and cold; and Radix Paeoniae Alba (dried root of Paeonia lactiflora Pall.) and Radix Paeoniae Rubra (dried root of Paeonia veitchii Lynch) are used for harmonizing the nutrient qi to alleviate pain. The whole prescription plays the function of warming Yang, dispersing cold, dehumidifying and relieving pain. However, the Radix Aconiti Lateralis Preparata in the prescription is extremely poisonous, and improper clinical application of Radix Aconiti Lateralis Preparata can not only produce toxic side effects, but may also cause patient death.

Aconitine, the toxic component of Radix Aconiti Lateralis Preparata, is soluble in both water and ethanol and is extremely toxic. Oral administration of 0.2 mg of the aconitine can cause poisoning reactions, and 3 mg to 5 mg of the aconitine can cause death. Therefore, Radix Aconiti Lateralis Preparata is classified as a lower grade product in the “Shen Nong's Materia Medica”. Since “poison” and “effect” coexist, Radix Aconiti Lateralis Preparata has both a toxic side and a therapeutic side. Improper use of it can cause toxic side effects, while correct use of Radix Aconiti Lateralis Preparata generally produces quick and efficient effects, treating many critical and serious diseases. As a result, some Chinese medicine practitioners call Radix Aconiti Lateralis Preparata a strong traditional Chinese medicine that patients cannot tolerate, and dare not use it casually. Accordingly, there are always potential risks in the clinical application of Radix Aconiti Lateralis Preparata, and the toxicity of Radix Aconiti Lateralis Preparata is the first clinical problem to be solved.

Early decoction is the main method to reduce the toxicity of Radix Aconiti Lateralis Preparata. In large doses, Radix Aconiti Lateralis Preparata is generally decocted for half an hour and tasted. After half an hour, when Radix Aconiti Lateralis Preparata no longer shows numb taste, other traditional Chinese medicines can be added to allow decoction together. However, the above-mentioned attenuation method still cannot guarantee the safety of clinical application of Radix Aconiti Lateralis Preparata, and no truly nontoxic Radix Aconiti Lateralis Preparata has been developed. The reason is that the clinical correlation between the toxicity, dosage, and processing methods of Radix Aconiti Lateralis Preparata is unclear, and there is a lack of unified standards for attenuated processing of Radix Aconiti Lateralis Preparata. The present disclosure is an application development result obtained from research on the above problems and has desirable application prospects.

### SUMMARY

In view of this, an objective of the present disclosure is to provide a toxicity-removed and medicinal effect-retained Fuzi decoction granule and a preparation method thereof. In the present disclosure, a medicinal effect of the Fuzi decoction granule can be retained on the basis of removing the toxicity of Radix Aconiti Lateralis Preparata.

To achieve the above objective, the present disclosure provides the following technical solutions:
The present disclosure provides a preparation method of a toxicity-removed and medicinal effect-retained Fuzi decoction granule, including the following steps: (1) subjecting Radix Aconiti Lateralis Preparata to immersion for 25 min to 35 min and decoction in water in a container for 60 min to 120 min; (2) mixing Poria, Radix Ginseng, bran-fried Rhizoma Atractylodis Macrocephalae, Radix Paeoniae Alba, Radix Paeoniae Rubra, and Ramulus Cinnamomi to allow immersion in water to obtain an immersed traditional Chinese medicine mixture; (3) adding the immersed traditional Chinese medicine mixture obtained in step (2) into the container for the decoction in step (1) to allow decoction with the Radix Aconiti Lateralis Preparata for 30 min to 60 min, and then conducting filtering to obtain a filtrate; (4) concentrating the filtrate obtained in step (3) to obtain a paste, mixing the paste with maltodextrin, and then subjecting a resulting mixture to spray-drying and granulation in sequence to obtain the toxicity-removed and medicinal effect-retained Fuzi decoction granule.

Preferably, the Radix Aconiti Lateralis Preparata, the Poria, the Radix Ginseng, the bran-fried Rhizoma Atractylodis Macrocephalae, the Radix Paeoniae Alba, the Radix Paeoniae Rubra, and the Ramulus Cinnamomi are at a mass ratio of 15:9:6:12:15:15:12.

Preferably, the Radix Aconiti Lateralis Preparata and the water in step (1) are at a mass ratio of 1:(8-12).

Preferably, a total mass of the Poria, the Radix Ginseng, the bran-fried Rhizoma Atractylodis Macrocephalae, the Radix Paeoniae Alba, the Radix Paeoniae Rubra, and the Ramulus Cinnamomi and a mass of the water in step (2) are at a ratio of 1:(8-12).

Preferably, the immersion in step (2) is conducted for 25 min to 35 min.

Preferably, the paste has a relative density of 1.02 to 1.12.

Preferably, the paste and the maltodextrin are at a mass ratio 1:0.3.

Preferably, the spray-drying is conducted at 165° C. to 190° C.

Preferably, the toxicity-removed and medicinal effect-retained Fuzi decoction granule has a particle size of 10 mesh to 16 mesh.

The present disclosure further provides a Fuzi decoction granule prepared by the preparation method, where the Fuzi decoction granule includes benzoyl hypaconitine greater than or equal to 43.35 μg/mg, benzoyl mesaconitine greater than or equal to 268.52 μg/mg, benzoyl aconitine greater than or equal to 28.75 μg/mg, hypaconitine less than or equal to 5.63 μg/mg, mesaconitine less than or equal to 0.05 μg/mg, and no aconitine.

Compared with the prior art, the present disclosure has the following beneficial effects:

The present disclosure provides a preparation method of a toxicity-removed and medicinal effect-retained Fuzi decoction granule, including the following steps: subjecting Radix Aconiti Lateralis Preparata to immersion and decoction in advance for 60 min to 120 min, and then mixing with Poria, Radix Ginseng, bran-fried Rhizoma Atractylodis Macrocephalae, Radix Paeoniae Alba, Radix Paeoniae Rubra, and Ramulus Cinnamomi to allow immersion for 30 min to 60 min; and subjecting a filtrate obtained by filtration to concentration and granulation in sequence to obtain the toxicity-removed and medicinal effect-retained Fuzi decoction granule. The Fuzi decoction granule prepared by the preparation method can remove inherent toxicity and retain a medicinal effect on osteoarthritis.

### BRIEF DESCRIPTION OF THE DRAWINGS

FIG. 1 shows the influence of Fuzi decoction granule on body weight of mice at different times (g, x±s); where FZ-30 is compared with a normal control group, *P<0.05, ** P<0.01; FZ-60 is compared with the normal control group, #P<0.05, ##P<0.01;

FIGS. 2A-2F show influences of each treatment on the blood biochemistry of mice in 2 weeks (g, x±s); where the FZ-30 is compared with the normal control group, *P<0.05, ** P<0.01;

FIGS. 3A-3F show influences of each treatment on the blood biochemistry of mice in 4 weeks (g, x±s); where the FZ-30 is compared with the normal control group, *P<0.05, ** P<0.01;

FIGS. 4A-4C show the statistical analysis and histopathological observation of Fuzi decoction granules in non-long time decoction and long time decoction on pain in rat OA model established by the method of monoiodoacetate; where FIG. 4A is the H&E staining and SO staining; scale bar=100 μm; FIG. 4B is the Mankin's score of histopathological observation; and FIG. 4C is the mechanical pains at 0 and 4 weeks; ##P<0.01 is comparing with the normal group; ** P<0.01 is comparing with the model group;

FIGS. 5A-5C show immunohistochemical analysis of the influences of Fuzi decoction granules in non-long time decoction and long time decoction on the expression of Col2 and MMP13 in rat cartilage; where FIG. 5A is the immunohistochemical staining of cartilage Col2 and MMP13, scale bar=100 μm; FIGS. 5B-5C are the quantitative examination of the percentage of Col2-positive area and the percentage of MMP13-positive cells; ##P<0.01 is comparing with the normal group; * P<0.05 is comparing with the model group; ** P<0.01 is comparing with the model group.

### DETAILED DESCRIPTION OF THE EMBODIMENTS

The present disclosure provides a preparation method of a toxicity-removed and medicinal effect-retained Fuzi decoction granule, including the following steps: (1) subjecting Radix Aconiti Lateralis Preparata to immersion for 25 min to 35 min and decoction in water in a container for 60 min to 120 min; (2) mixing Poria, Radix Ginseng, bran-fried Rhizoma Atractylodis Macrocephalae, Radix Paeoniae Alba, Radix Paeoniae Rubra, and Ramulus Cinnamomi to allow immersion in water to obtain an immersed traditional Chinese medicine mixture; (3) adding the immersed traditional Chinese medicine mixture obtained in step (2) into the container for the decoction in step (1) to allow decoction with the Radix Aconiti Lateralis Preparata for 30 min to 60 min, and then conducting filtering to obtain a filtrate; (4) concentrating the filtrate obtained in step (3) to obtain a paste, mixing the paste with maltodextrin, and then subjecting a resulting mixture to spray-drying and granulation in sequence to obtain the toxicity-removed and medicinal effect-retained Fuzi decoction granule.

In the present disclosure, the Radix Aconiti Lateralis Preparata is a prepared Radix Aconiti Lateralis Preparata, which preferably includes Heishunpian, Baifupian, Huangfupian, Yanfupian, and Danfupian. The decoction of Radix Aconiti Lateralis Preparata in step (1) is conducted for preferably 90 min, and the decoction in step (3) is conducted for preferably 30 min. The decoction in the present disclosure can remove the toxicity in Radix Aconiti Lateralis Preparata and retain the medicinal effect on osteoarthritis.

In the present disclosure, the Radix Aconiti Lateralis Preparata, the Poria, the Radix Ginseng, the bran-fried Rhizoma Atractylodis Macrocephalae, the Radix Paeoniae Alba, the Radix Paeoniae Rubra, and the Ramulus Cinnamomi are at a mass ratio of preferably 15:9:6:12:15:15:12; the Radix Aconiti Lateralis Preparata and the water in step (1) are at a mass ratio of preferably 1:(8-12), more preferably 1:10; a total mass of the Poria, the Radix Ginseng, the bran-fried Rhizoma Atractylodis Macrocephalae, the Radix Paeoniae Alba, the Radix Paeoniae Rubra, and the Ramulus Cinnamomi and a mass of the water in step (2) are at a ratio of preferably 1:(8-12), more preferably 1:10; the immersion in step (2) is conducted for preferably 25 min to 35 min; the paste has a relative density of preferably 1.02 to 1.12, more preferably 1.08.

In the present disclosure, the paste and the maltodextrin are at a mass ratio of preferably 1:0.3; the spray-drying is conducted at preferably 165° C. to 190° C., more preferably 180° C.; the toxicity-removed and medicinal effect-retained Fuzi decoction granule has a particle size of preferably 10 mesh to 16 mesh, more preferably 14 mesh; packaging is preferably conducted after the granulation is completed.

In the present disclosure, there are no special restrictions on sources of the raw materials, as long as conventional commercial products in the art may be used.

The present disclosure further provides a Fuzi decoction granule prepared by the preparation method, where the Fuzi decoction granule includes benzoyl hypaconitine greater than or equal to 43.35 μg/mg, benzoyl mesaconitine greater than or equal to 268.52 μg/mg, benzoyl aconitine greater than or equal to 28.75 μg/mg, hypaconitine less than or equal to 5.63 μg/mg, mesaconitine less than or equal to 0.05 μg/mg, and no aconitine.

In the present disclosure, there are no special restrictions on mechanical equipment used in the process of preparing the Fuzi decoction granule, and conventional equipment in this field can be used. The concentration is conducted using a small extraction and concentration unit, model 100 L; the drying is conducted using a centrifugal spray dryer, model ZPG-5; the mixing is conducted using a three-dimensional motion mixer, model SYH-1000; the granulation is conducted using a dry granulator, model LGJ80; the packaging is conducted using an automatic granule packaging machine, model DXDK40VI.

The technical solution provided by the present disclosure will be described in detail below with reference to the examples, but they should not be construed as limiting the claimed scope of the present disclosure.

### Example 1

(1) 15 g of Radix Aconiti Lateralis Preparata (Heishunpian) was immersed in 10 times water for 30 min and decocted for 90 min;

(2) 9 g of Poria, 6 g of Radix Ginseng, 12 g of bran-fried Rhizoma Atractylodis Macrocephalae, 15 g of Radix Paeoniae Alba, 15 g of Radix Paeoniae Rubra, and 12 g of Ramulus Cinnamomi were mixed to allow immersion in 10 times water for 30 min, and filtered to obtain an immersed traditional Chinese medicine mixture;

(3) the immersed traditional Chinese medicine mixture obtained in step (2) was added into the container for the decoction in step (1) to allow decoction with the Radix Aconiti Lateralis Preparata for 30 min, and then filtered to obtain a filtrate;

(4) the filtrate obtained in step (3) was concentrated to obtain a paste with a relative density of 1.08, the paste was mixed with 25.2 g of maltodextrin, and then a resulting mixture was subjected to spray-drying at an inlet temperature of 165° C. to 190° C. and an outlet temperature of 70° C. to 90° C. and granulation at 14 mesh in sequence to obtain the toxicity-removed and medicinal effect-retained Fuzi decoction granule, which was packaged at 10 g per package.

### Example 2

(1) 15 g of Radix Aconiti Lateralis Preparata (Heishunpian) was immersed 8 times water for 35 min and decocted for 60 min;

(2) 9 g of Poria, 6 g of Radix Ginseng, 12 g of bran-fried Rhizoma Atractylodis Macrocephalae, 15 g of Radix Paeoniae Alba, 15 g of Radix Paeoniae Rubra, and 12 g of Ramulus Cinnamomi were mixed to allow immersion in 8 times water for 35 min, and filtered to obtain an immersed traditional Chinese medicine mixture;

(3) the immersed traditional Chinese medicine mixture obtained in step (2) was added into the container for the decoction in step (1) to allow decoction with the Radix Aconiti Lateralis Preparata for 60 min, and then filtered to obtain a filtrate;

(4) the filtrate obtained in step (3) was concentrated to obtain a paste with a relative density of 1.02, the paste was mixed with 25.2 g of maltodextrin, and then a resulting mixture was subjected to spray-drying at an inlet temperature of 165° C. to 190° C. and an outlet temperature of 70° C. to 90° C. and granulation at 14 mesh in sequence to obtain the toxicity-removed and medicinal effect-retained Fuzi decoction granule, which was packaged at 10 g per package.

### Example 3

(1) 15 g of Radix Aconiti Lateralis Preparata (Heishunpian) was immersed 12 times water for 25 min and decocted for 120 min;

(2) 9 g of Poria, 6 g of Radix Ginseng, 12 g of bran-fried Rhizoma Atractylodis Macrocephalae, 15 g of Radix Paeoniae Alba, 15 g of Radix Paeoniae Rubra, and 12 g of Ramulus Cinnamomi were mixed to allow immersion in 8 times water for 25 min, and filtered to obtain an immersed traditional Chinese medicine mixture;

(3) the immersed traditional Chinese medicine mixture obtained in step (2) was added into the container for the decoction in step (1) to allow decoction with the Radix Aconiti Lateralis Preparata for 30 min, and then filtered to obtain a filtrate;

(4) the filtrate obtained in step (3) was concentrated to obtain a paste with a relative density of 1.12, the paste was mixed with 25.2 g of maltodextrin, and then a resulting mixture was subjected to spray-drying at an inlet temperature of 165° C. to 190° C. and an outlet temperature of 70° C. to 90° C. and granulation at 14 mesh in sequence to obtain the toxicity-removed and medicinal effect-retained Fuzi decoction granule, which was packaged at 10 g per package.

### Comparative Example 1

This comparative example was the same as Example 1, except that the decoction in step (1) was conducted for 0 min, and the decoction in step (3) was conducted for 30 min.

### Comparative Example 2

This comparative example was the same as Example 1, except that the decoction in step (1) was conducted for 30 min, and the decoction in step (3) was conducted for 30 min.

### Comparative Example 3

(1) 15 g of Radix Aconiti Lateralis Preparata (Heishunpian), 9 g of Poria, 6 g of Radix Ginseng, 12 g of bran-fried Rhizoma Atractylodis Macrocephalae, 15 g of Radix Paeoniae Alba, 15 g of Radix Paeoniae Rubra, and 12 g of Ramulus Cinnamomi were mixed to allow immersion in 10 times water for 30 min and decoction for 30 min, and filtered to obtain an immersed traditional Chinese medicine mixture;

(2) the filtrate obtained in step (2) was concentrated to obtain a paste with a relative density of 1.08, the paste was mixed with 25.2 g of maltodextrin, and then a resulting mixture was subjected to spray-drying at an inlet temperature of 165° C. to 190° C. and an outlet temperature of 70° C. to 90° C. and granulation at 14 mesh in sequence to obtain the toxicity-removed and medicinal effect-retained Fuzi decoction granule, which was packaged at 10 g per package.

### Experimental Example 1

The contents of three toxic alkaloids and three functional alkaloids in the Fuzi decoction granules of Example 1 and Comparative Example 3 were determined.

A detection method was as follows:

### 1. Preparation of Reference Solutions

The standard substances of functional aconitines (benzoyl hypaconitine, benzoyl mesaconitine, and benzoyl aconitine) and toxic aconitines (hypaconitine, mesaconitine, and aconitine) were precisely weighed, diluted with dichloromethane (DCM), and dissolved by shaking well to obtain a reference mixed solution.

### 2. Preparation of Test Solution

18 mL of an aqueous solution (with a concentration of 2.0 g/mL) for each of the Fuzi decoction granules in Example 1 and Comparative Example 3 were taken, and extracted 3 times separately with DCM and chloroform, 18 mL/time. Obtained extracts of the DCM and chloroform layers were combined, recovered under reduced pressure at 55° C. until dryness, diluted to 3 mL with DCM, and filtered with a 0.45 μm microporous filter to obtain the test solution.

### 3. Analysis and Determination by UPLC-MS/MS

Chromatographic conditions were as follows:

Chromatographic column: BEH C18 2.1 mm*100 mm, 1.7 um; mobile phase A: 5 mM ammonium formate aqueous solution, mobile phase B: acetonitrile; flow rate: 1.0 mL/min, column temperature 30° C., and injection volume 5 μL.

The content of the active ingredients in the reference solution was measured to formulate a standard curve. According to the standard curve, the contents of the 6 ingredients in the Fuzi decoction granules of Example 1 and Comparative Example 3 were calculated. The specific results were shown in Table 1.

Table 1 Contents of 3 toxic alkaloids and 3 functional alkaloids in Fuzi decoction granules

| | Benzoyl | Benzoyl | Benzoyl | | | |
|---|---|---|---|---|---|---|
| | hypaconitine | mesaconitine | aconitine | Hypaconitine | Mesaconitine | Aconitine |
| | μg/g | (μg/g) | μg/g | μg/g | μg/g | μg/g |
| Comparative | 10.22 ± 0.00 | 65.88 ± 0.01 | 10.25 ± 0.01 | 8.51 ± 0.02 | 0.49 ± 0.00 | 0.31 ± 0.01 |
| Example 3 | | | | | | |
| Example 1 | 43.35 ± 0.03 | 268.52 ± 0.03 | 28.75 ± 0.04 | 5.63 ± 0.02 | 0.05 ± 0.17 | ND |

As shown in Table 1, compared to Comparative Example 3, the Fuzi decoction granule prepared by the method of Example 1 could significantly increase the contents of functional aconitines and reduce the contents of toxic aconitines.

### Experimental Example 2

ICR mouse acute toxicity test
1. Experimental animals: 40 ICR mice, half male and half male, 25-30 g, provided by the Animal Center of Zhejiang Chinese Medical University.
2. Solution of Fuzi decoction granule:
50 g of Fuzi decoction granules from Example 1 and Comparative Examples 1 to 2 were taken (1 g of the granule was equivalent to 3 g of a crude drug), dissolved in water, and diluted to 150 mL to obtain a crude drug with a concentration of 1 g/mL.
3. Grouping and administration:
40 clean-grade ICR mice were randomly divided into groups according to body weight: a normal control group, a Fuzi decoction granule (decoction of Radix Aconiti Lateralis Preparata in water for 30 min) group (Comparative Example 1), a Fuzi decoction granule (decoction of Radix Aconiti Lateralis Preparata in water for 60 min) group (Comparative Example 2), and a Fuzi decoction granule (decoction of Radix Aconiti Lateralis Preparata in water for 120 min) group (Example 1), 10 animals in each group, half male and half female. The mice were fasted and given water for 12 h before the experiment. The mice in each group were administered the maximum intragastric dose (0.02 mL/g/d) once a day for 28 consecutive days, while the mice in normal control group was given distilled water every day.
4. Daily weighing of mice and observation of conditions after intragastric administration:

### (1) Observation

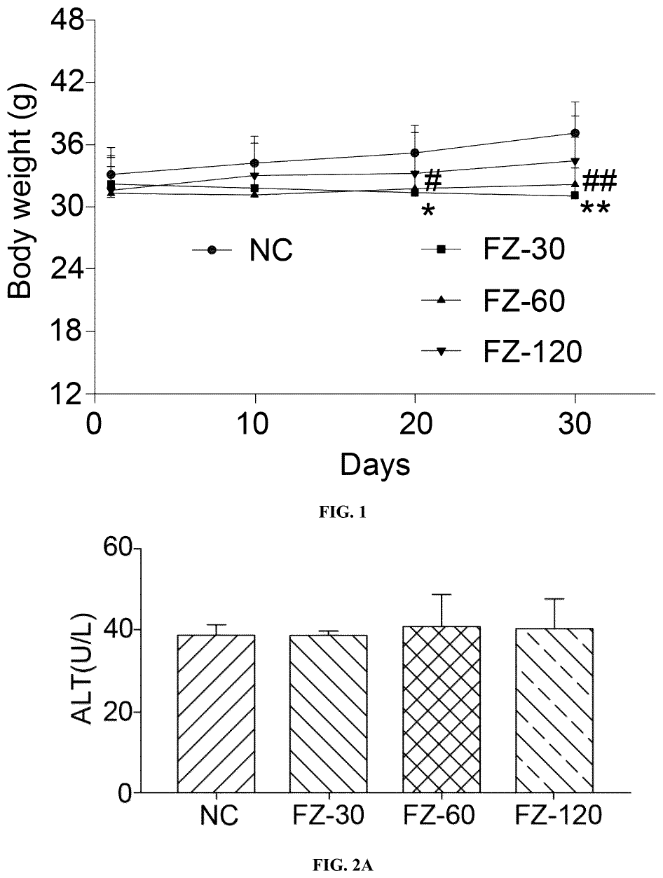

During the dosing period (28 d), the mice in each group were observed every day for changes in body weight (FIG. 1 for specific results), diet, appearance, behavior, secretions, excretions, death, and poisoning reactions (including symptoms, severity, starting time, duration, and whether the poisoning reaction was reversible).

As shown in FIG. 1, compared with the normal control group, the body weight of mice in each administration group decreased significantly (P<0.05); by comparing the body weight differences, it was seen that there was a significant difference in the toxicity of Fuzi decoction granule to mice with different decoction times of Radix Aconiti Lateralis Preparata (P<0.05), indicating that the toxicity of Fuzi decoction granule to mice decreased with the delay of decoction time.

The mice in the normal control group were active, walking back and forth in the cage, and eating and drinking normally. The mice in the Fuzi decoction granule (decoction of Radix Aconiti Lateralis Preparata in water for 30 min) group showed symptoms such as rapid breathing, restlessness, and then closing of eyes, cloudy and dim eyeballs, depression, shortness of breath, low body temperature, laziness, and poor coordination. There were no significant changes in the mice in the Fuzi decoction granule (decoction of Radix Aconiti Lateralis Preparata in water for 60 min) and Fuzi decoction granule (decoction of Radix Aconiti Lateralis Preparata in water for 120 min) groups, and their activity levels decreased.

### (2) Blood Test

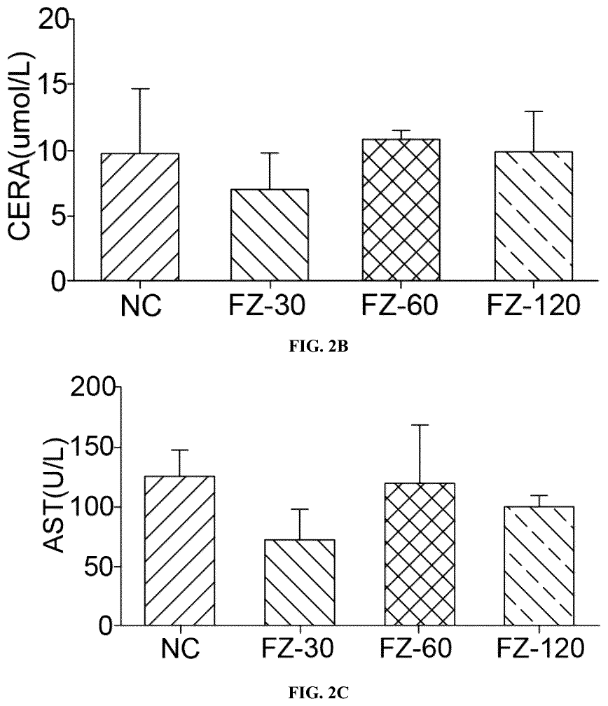

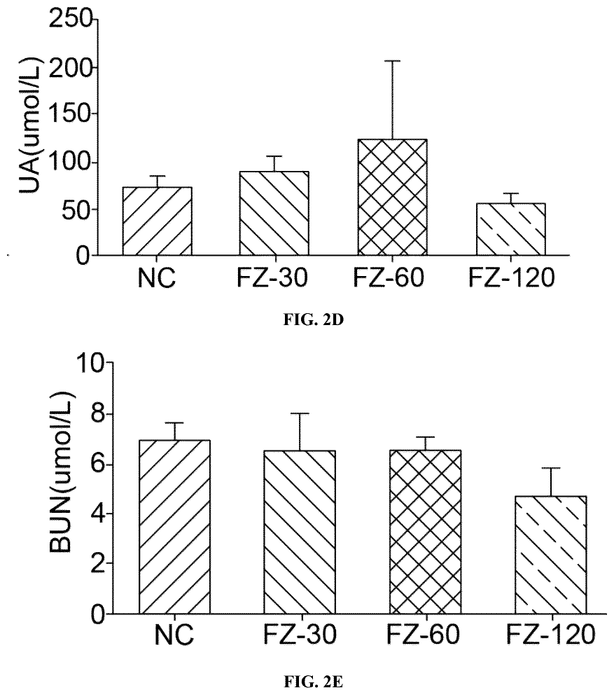

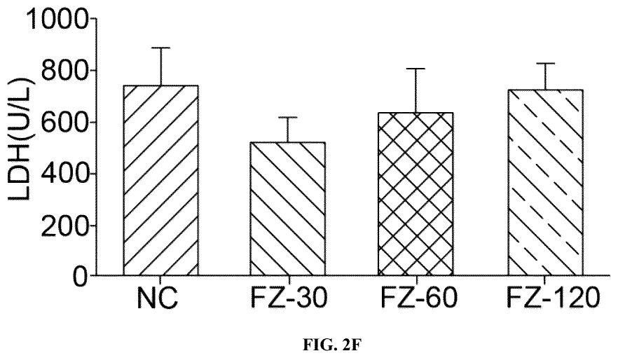

After the 28-d observation period, blood samples were taken from the surviving mice in each group, the blood samples were allowed to stand for 2 h and centrifuged at low speed of 3,000 r/min for 10 min, and a resulting supernatant was taken to obtain the mouse serum. The following indicators in the serum were detected using relevant instruments and equipment, including: alanine aminotransferase (ALT), aspartate aminotransferase (AST), blood urea nitrogen (BUN), uric acid (UA), creatinine (CREA), and lactate dehydrogenase (LDH). The specific results were shown in Tables 2 to 3 and FIGS. 2A-2F.

TABLE 2
White blood cell and differential examination results of mice fed
Fuzi decoction granule for 28 d under different decoction times of
Radix Aconiti Lateralis Preparata (x̅ ± s)

| | | White blood | | |
|---|---|---|---|---|
| | Quantity | cell count | Lymphocyte | Neutrophil |
| Group | (mouse) | (10⁹ cells/L) | (%) | (%) |
| Control | 10 | 5.15 ± 0.51 | 81.9 ± 1.62 | 16.12 ± 1.53 |
| Fuzi decoction | 10 | 10.72 ± 0.50** | 41.9 ± 4.10** | 53.7 ± 7.00** |
| granule (decoction of | | | | |
| Radix Aconiti | | | | |
| Lateralis Preparata | | | | |
| in water for 30 min) | | | | |
| Fuzi decoction | 10 | 5.46 ± 1.00 | 72.74 ± 0.70** | 24.32 ± 1.00** |
| granule (decoction of | | | | |
| Radix Aconiti | | | | |
| Lateralis Preparata | | | | |
| in water for 60 min) | | | | |
| Fuzi decoction | 10 | 5.11 ± 0.46 | 83.15 ± 3.77 | 15.02 ± 3.54 |
| granule (decoction of | | | | |
| Radix Aconiti | | | | |
| Lateralis Preparata | | | | |
| in water for 120 min) | | | | |

**represented a significant difference.

TABLE 3
Red blood cell and platelet test results of mice fed Fuzi decoction granule for 28 d under
different decoction times of Radix Aconiti Lateralis Preparata (x̅ ± s)

| | | Total number of | | | |
|---|---|---|---|---|---|
| | Quantity | red blood cells | Hemoglobin | Hematocrit | Platelet |
| Group | (mouse) | (10¹² cells/L) | (g/L) | (%) | (10⁹ cells/L) |
| Control | 10 | 9.83 ± 0.61 | 136.83 ± 7.68 | 45.15 ± 2.14 | 1788.17 ± 257.42 |
| Decoction of Radix | 10 | 9.11 ± 0.33 | 126.00 ± 7.36 | 41.10 ± 1.15 | 352.00 ± 505.37** |
| Aconiti Lateralis | | | | | |
| Preparata in water | | | | | |
| for 30 min | | | | | |
| Decoction of Radix | 10 | 29.80 ± 0.47** | 138.40 ± 7.36 | 45.36 ± 2.58 | 1643.60 ± 362.54 |
| Aconiti Lateralis | | | | | |
| Preparata in water | | | | | |
| for 60 min | | | | | |
| Decoction of Radix | 10 | 9.64 ± .65 | 133.16 ± 7.54 | 43.82 ± 2.71 | 1560.33 ± 257.49 |
| Aconiti Lateralis | | | | | |
| Preparata in water | | | | | |
| for 120 min | | | | | |

As shown in Tables 2 to 3, compared with the normal control group, the white blood cells, lymphocytes, and neutrophils in the blood of the mice in the decoction of Radix Aconiti Lateralis Preparata in water for 30 min group were all increased, and the difference was statistically significant (P<0.05), indicating that the Fuzi decoction granule (decoction of Radix Aconiti Lateralis Preparata in water for 30 min) could cause some inflammation in mice. Compared with those in the normal control group, lymphocytes and neutrophils in the Fuzi decoction granule (decoction of Radix Aconiti Lateralis Preparata in water for 60 min) and Fuzi decoction granule (decoction of Radix Aconiti Lateralis Preparata in water for 120 min) showed varying degrees of abnormal changes (P<0.05). The total number of red blood cells and platelets also showed abnormal changes in the Fuzi decoction granule (decoction of Radix Aconiti Lateralis Preparata in water for 30 min) and Fuzi decoction granule (decoction of Radix Aconiti Lateralis Preparata in water for 60 min) groups (P<0.05), but there was no change in the above indicators in the Fuzi decoction granule (decoction of Radix Aconiti Lateralis Preparata in water for 120 min) group (P>0.05).

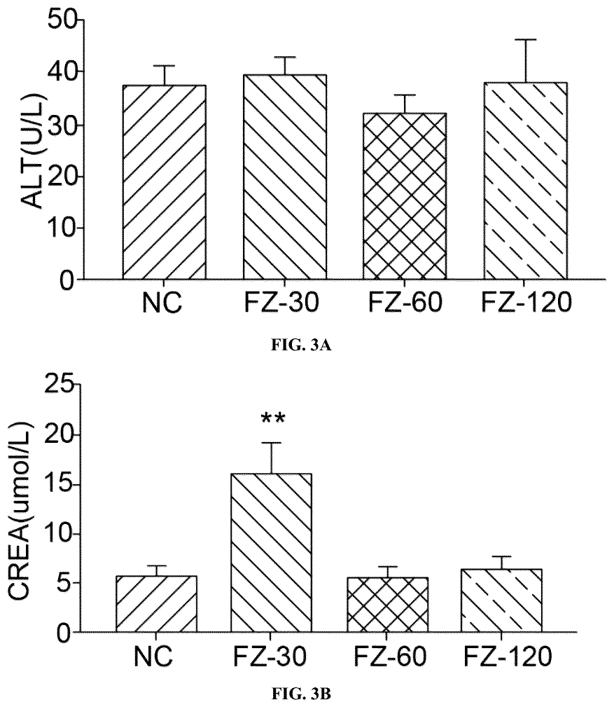

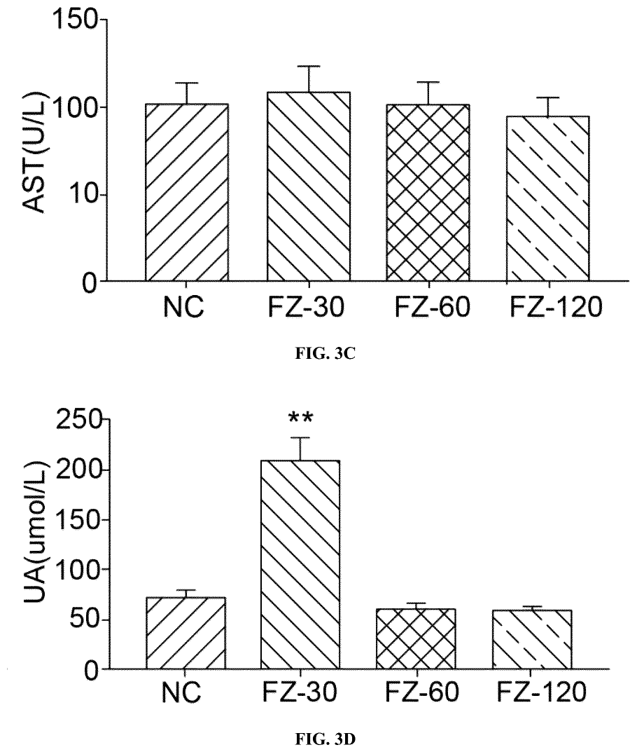

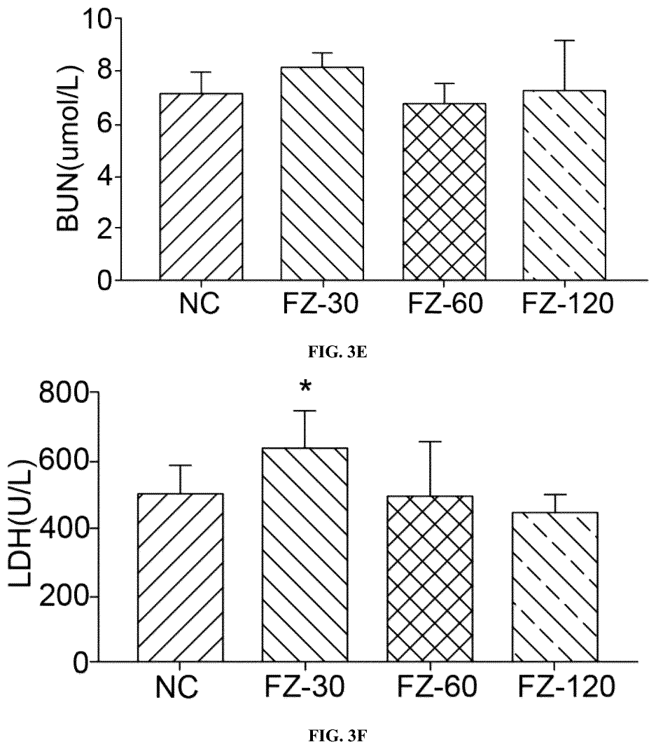

As shown in FIGS. 2A-2F and FIGS. 3A-3F, compared with those in the normal control group, the levels of ALT, GOT, CK, and LDH in the blood of mice in the Fuzi decoction granule (decoction of Radix Aconiti Lateralis Preparata in water for 30 min) group were all increased and the differences were statistically significant (P<0.05), indicating that the Fuzi decoction granule (decoction of Radix Aconiti Lateralis Preparata in water for 30 min) had certain toxicity to the liver and heart. However, there were no significant differences in the ALT, AST, CK, and LDH contents in the Fuzi decoction granule (decoction of Radix Aconiti Lateralis Preparata in water for 60 min and 120 min) group compared with those in the normal control group (P>0.05).

The above results showed that the Fuzi decoction granule (decoction of Radix Aconiti Lateralis Preparata in water for 120 min) group was safer than other groups.

### Experimental Example 3

Therapeutic effect of the Fuzi decoction granule on osteoarthritis rat

### 1. Experimental Grouping

According to the random number table method, 40 SD rats were randomly divided into 4 groups (10 rats in each group): a sham operation group, a model group, a Fuzi decoction granule group with non-long time decoction (Comparative Example 3), and Fuzi decoction granule group with long time decoction (Example 1).

### 2. Modeling

Except for the sham operation group, the other groups used the method of injecting iodoacetic acid into the joint cavity to establish kOA models. The rats were anesthetized with intraperitoneal injection of 3% sodium pentobarbital (0.15 ml/100 g). After the rats were anesthetized, their local hair of the knee joints was shaved, disinfected with iodophor, and the rats were fixed and their knee joints were flexed. 50 μL of the iodoacetic acid (20 mg/mL) was injected into the unilateral knee joint cavity with a microsyringe to obtain an osteoarthritis (OA) model rat.

### 3. Treatment

After modeling, the rats in Fuzi decoction granule group (non-long time decoction)/Fuzi decoction granule group (long time decoction) were orally gavaged with 5.4 g/kg/d, while the rats in sham operation group and model group were orally gavaged with a same mass of normal saline; the treatment lasted for 30 d.

### 4. Pain Statistical Analysis and Histopathological Observation of Rat OA Model

Pain behavioral testing: mechanical hypersensitivity was measured using the “up-down” test method with a series of von Frey filaments (ranging from 0.6-26 g, UGO Basile, Italy). Briefly, the von Frey filaments were pressed vertically against the mid-metatarsal surface of the hind paw with sufficient force to cause the metatarsal to flex, and 50% PWT was calculated.

Material collection: after the experiment, the rats were anesthetized by intraperitoneal injection of sodium pentobarbital (3% concentration, 0.15 mL/100 g), and blood was collected from the heart using a 10 mL syringe. The muscles near the knee joint were removed with scissors, and the knee joint was removed with rongeurs and fixated in 4% paraformaldehyde solution for 3 d. After fixation for 3 d, the paraformaldehyde on the surface of the joint sample was washed with running water, the sample was placed into an embedding box, marked, and placed in EDTA solution for decalcification. An EDTA decalcification solution was changed every day and the decalcification was continued for about 2 months. When the bone tissue in the joint softened, the samples were rinsed under running water overnight. The samples were dehydrated in a dehydrator and finally embedded in paraffin. Each sample was cut into 3 μm thick sections using a microtome for subsequent staining.

Staining: the tissue sections were baked in a 60° C. oven overnight to prevent sections from falling off; the sections were placed in 3 cylinders of xylene for dewaxing, 10 min in each cylinder; the sections were subjected to gradient alcohol rehydration (alcohol concentration was 100%, 100%, 95%, 95%, 70% sequentially in each tank), 5 min in each tank; the sections were allowed to stand in pure water for 3 min. The H&E staining specifically included: dewaxing and rehydration; hematoxylin for 2 min, rinsing with pure water for 3 min/time, three times in total; decomposing with 1% hydrochloric acid alcohol for 2-3 s, rinsing with pure water for 3 min/time, three times in total; re-bluing with 0.5% ammonia water for 10 s, rinsing with pure water for 3 min/time, three times in total; eosin for 1 min, rinsing with pure water 3 min/time, three times in total; immersing in 95% ethanol, 100% ethanol, xylene I, xylene II, and xylene III in sequence for 1 min, dehydrating to allow vitrification, and sealing with neutral resin. The SO staining specifically included: dewaxing and rehydration; fast green dyeing solution for 5 min, rinsing with pure water for 3 min/time, three times in total; decomposing with 1% glacial acetic acid for 10 s, rinsing with pure water for 3 min/time, three times in total; safranine O dyeing solution for 1 min, rinsing with pure water for 3 min/time, three times in total; after decomposition with 95% ethanol for a few seconds, rinsing with pure water for 3 min/time, three times in total; drying in an oven at 37° C. and then placing in xylene to allow vitrification, and sealing with neutral resin.

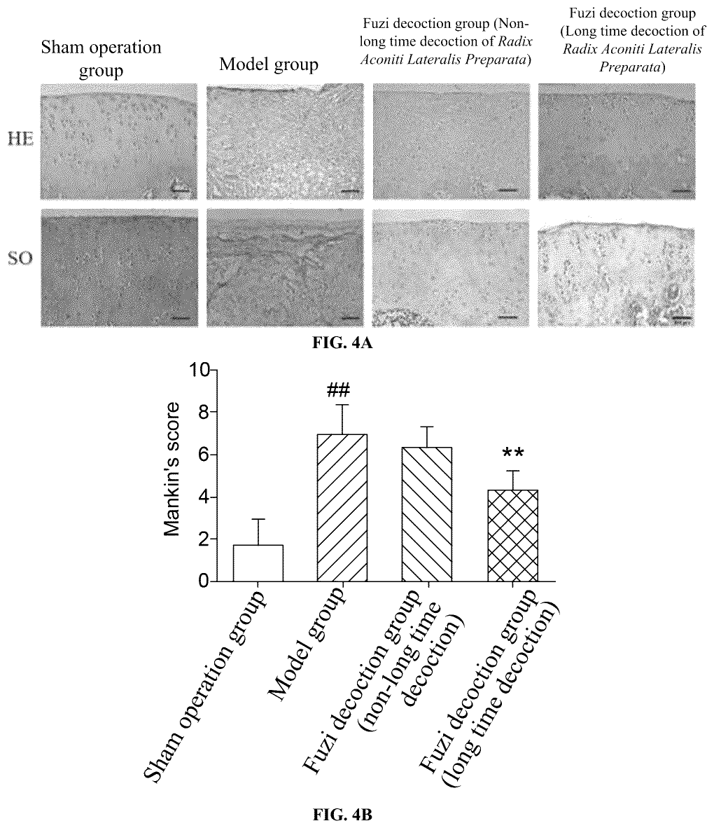

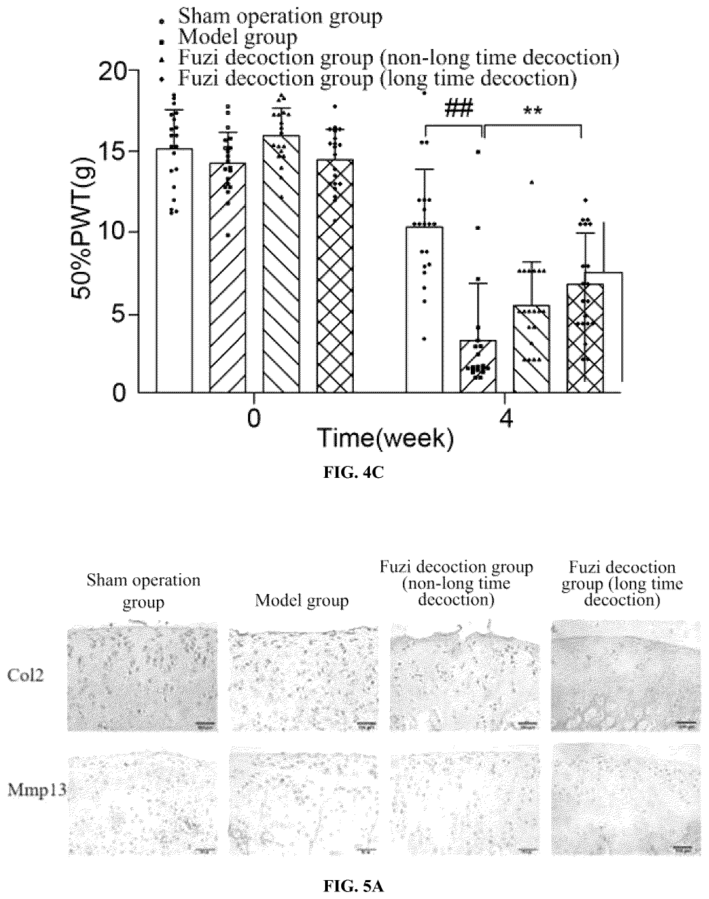

The pain statistical analysis and histopathological observations were shown in FIGS. 4A-4C.

As shown in FIGS. 4A-4C, histopathological staining indicated significant degeneration of cartilage, reduction and apoptosis of chondrocytes, massive destruction of collagen, and matrix disorder; while pain behavior test results indicated that the OA rats had more sensitive pain responses. The above results all showed typical OA phenotypes. Compared with the normal group, the number of chondrocytes and the content of glycosaminoglycans (an important component of cartilage matrix) in the model group were significantly reduced, and the Mankin's score was also significantly increased (P<0.01) (FIG. 4B). In addition, the mechanical pain threshold (reflected by MWT) in the model group decreased significantly (P<0.01 compared with NC).

### 5. Immunohistochemistry

The joint tissue sections of rats in each group were dewaxed, rehydrated, and washed with PBS (1×) for 3 min/time, three times in total; sodium citrate solution was added for antigen retrieval, in a 60° C. oven for 4 h; the dye vat was taken out and rewarmed at room temperature for 30 min, and washed with PBS (1×) for 3 min each time for a total of three times; the sections were added into 0.1% Triton solution to punch the tissue, and washed with PBS (1×) for 3 min/time, three times in total; the PBS around the tissue was carefully removed, and a circle was drawn with a water-blocking pen around the tissue to fix the range of subsequent reagents; the PBS around the tissue was carefully removed, an appropriate amount of endogenous peroxidase blocker was added, incubated at room temperature for 10 min, and washed with PBS (1×) for 3 min/time, three times in total; 100 μL of primary antibody (Col2, MMP13, 1:100) diluted in PBS was added dropwise to each section, incubated at 4° C. overnight, and washed with PBS (1×) for 3 min/time, three times in total; the PBS around the tissue was carefully removed, about 50 μL of secondary antibody (enhanced enzyme-labeled goat anti-rabbit/anti-mouse IgG polymer), incubated at room temperature for 20 min, and washed with PBS (1×) for 3 min/time, three times in total; the PBS around the tissue was removed, an appropriate amount of freshly prepared DAB chromogenic solution was added, incubated at room temperature for 5-8 min, and washed with PBS (1×) for 3 min/time, three times in total; where the staining included: hematoxylin for 2 min, rinsing with pure water for 3 min/time, three times in total; decomposing with 1% hydrochloric acid alcohol for 2-3 s, rinsing with pure water for 3 min/time, three times in total; re-bluing with 0.5% ammonia water for 10 s, rinsing with pure water for 3 min/time, three times in total; immersing the sections in 95% ethanol, 100% ethanol, xylene I, xylene II, and xylene III in sequence for 1 min, dehydrated to allow vitrification, and sealed with neutral resin. After sealing, the sections were ventilated in a fume hood on a rack for several hours. After the neutral resin solidified and the xylene volatilized, the sections were photographed under an optical microscope. MMP13 expression level was quantified by counting the number of positive cells, and Col2 expression level was quantified by counting the positive area. A ratio of the antigen-positive area of the selected region/cell mass to the total area/total cell mass was used as an indicator for the final statistics.

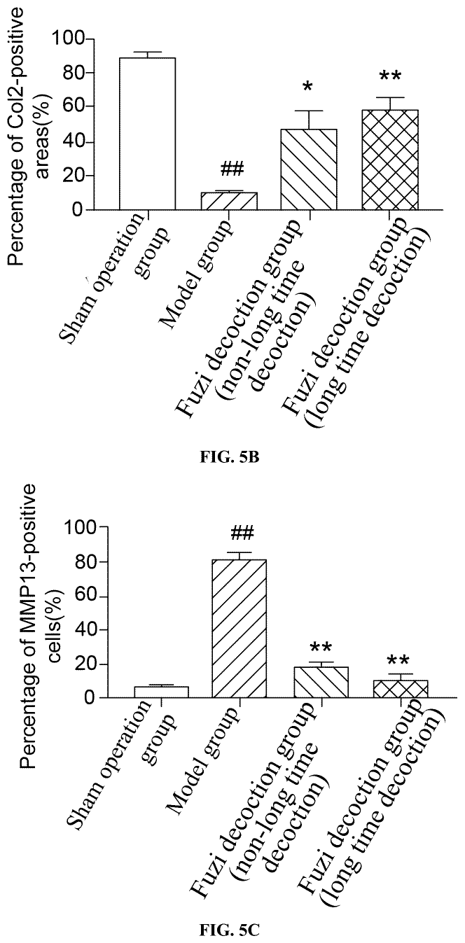

The immunohistochemistry results were shown in FIGS. 5A-5C.

As shown in FIGS. 5A-5C, in comparison, rats treated with the Fuzi decoction granule showed a significant increase in the number of chondrocytes and a decrease in apoptotic chondrocytes. Moreover, the Fuzi decoction granule improved the structural integrity of articular cartilage and the synthesis of glycosaminoglycans, and alleviated pain sensitization in OA rats. Furthermore, the effect of Fuzi decoction obtained after long-time decoction of Radix Aconiti Lateralis Preparata was better than that of Fuzi decoction obtained after non-long time decoction of Radix Aconiti Lateralis Preparata. These results were also expressed through the Mankin's score. The Mankin's score of the rats in the model group was significantly higher than that of the normal group; the Mankin's score of the Fuzi decoction granule group was significantly lower than that of the model group; and the effect of Fuzi decoction obtained after long-time decoction of Radix Aconiti Lateralis Preparata was better than that of Fuzi decoction obtained after non-long time decoction of Radix Aconiti Lateralis Preparata. The results of immunohistochemistry showed that the main component of cartilage matrix (Col2) and the cartilage matrix decomposition marker (MMP13) had abnormal changes in the model group; after treatment with Fuzi decoction granule, the expression of these markers significantly returned to normal levels; and the effect of Fuzi decoction obtained after long-time decoction of Radix Aconiti Lateralis Preparata was better than that of Fuzi decoction obtained after non-long time decoction of Radix Aconiti Lateralis Preparata. The above results indicated that the Fuzi decoction granule had a successful therapeutic effect on OA rats, and the effect of Fuzi decoction obtained after long-time decoction of Radix Aconiti Lateralis Preparata was better than that of Fuzi decoction obtained after non-long time decoction of Radix Aconiti Lateralis Preparata. Moreover, this protective effect might be achieved by inhibiting chondrocyte catabolismand chondrocyte hypertrophy and promoting chondrocyte anabolism.

In summary, it was seen that the technical solution of the present disclosure for preparing the Fuzi decoction granule by long time decoction of Radix Aconiti Lateralis Preparata was less toxic and more effective in treating osteoarthritis than that prepared by non-long time decoction of Radix Aconiti Lateralis Preparata.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A preparation method of a toxicity-removed and medicinal effect-retained Fuzi decoction granule, comprising the following steps:
(1) subjecting Radix Aconiti Lateralis Preparata to immersion for 25 min to 35 min and decoction in water in a container for 60 min to 120 min;
(2) mixing Poria, Radix Ginseng, bran-fried Rhizoma Atractylodis Macrocephalae, Radix Paeoniae Alba, Radix Paeoniae Rubra, and Ramulus Cinnamomi to allow immersion in water to obtain an immersed traditional Chinese medicine mixture;
(3) adding the immersed traditional Chinese medicine mixture obtained in step (2) into the container for the decoction in step (1) to allow decoction with the Radix Aconiti Lateralis Preparata for 30 min to 60 min, and then conducting filtering to obtain a filtrate;
(4) concentrating the filtrate obtained in step (3) to obtain a paste, mixing the paste with maltodextrin, and then subjecting a resulting mixture to spray-drying and granulation in sequence to obtain the toxicity-removed and medicinal effect-retained Fuzi decoction granule.

2. The preparation method according to claim 1, wherein the Radix Aconiti Lateralis Preparata, the Poria, the Radix Ginseng, the bran-fried Rhizoma Atractylodis Macrocephalae, the Radix Paeoniae Alba, the Radix Paeoniae Rubra, and the Ramulus Cinnamomi are at a mass ratio of 15:9:6:12:15:15:12.

3. The preparation method according to claim 1, wherein the Radix Aconiti Lateralis Preparata and the water in step (1) are at a mass ratio of 1:(8-12).

4. The preparation method according to claim 1, wherein a total mass of the Poria, the Radix Ginseng, the bran-fried Rhizoma Atractylodis Macrocephalae, the Radix Paeoniae Alba, the Radix Paeoniae Rubra, and the Ramulus Cinnamomi and a mass of the water in step (2) are at a ratio of 1:(8-12).

5. The preparation method according to claim 4, wherein the immersion in step (2) is conducted for 25 min to 35 min.

6. The preparation method according to claim 1, wherein the paste has a relative density of 1.02 to 1.12.

7. The preparation method according to claim 1, wherein the paste and the maltodextrin are at a mass ratio of 1:0.3.

8. The preparation method according to claim 1, wherein the spray-drying is conducted at 165° C. to 190° C.

9. The preparation method according to claim 1, wherein the toxicity-removed and medicinal effect-retained Fuzi decoction granule has a particle size of 10 mesh to 16 mesh.

10. A Fuzi decoction granule prepared by the preparation method according to claim 1, wherein the Fuzi decoction granule comprises benzoyl hypaconitine greater than or equal to 43.35 μg/mg, benzoyl mesaconitine greater than or equal to 268.52 μg/mg, benzoyl aconitine greater than or equal to 28.75 μg/mg, hypaconitine less than or equal to 5.63 μg/mg, mesaconitine less than or equal to 0.05 μg/mg, and no aconitine.

11. The Fuzi decoction granule according to claim 10, wherein the Radix Aconiti Lateralis Preparata, the Poria, the Radix Ginseng, the bran-fried Rhizoma Atractylodis Macrocephalae, the Radix Paeoniae Alba, the Radix Paeoniae Rubra, and the Ramulus Cinnamomi are at a mass ratio of 15:9:6:12:15:15:12.

12. The Fuzi decoction granule according to claim 10, wherein the Radix Aconiti Lateralis Preparata and the water in step (1) are at a mass ratio of 1:(8-12).

13. The Fuzi decoction granule according to claim 10, wherein a total mass of the Poria, the Radix Ginseng, the bran-fried Rhizoma Atractylodis Macrocephalae, the Radix Paeoniae Alba, the Radix Paeoniae Rubra, and the Ramulus Cinnamomi and a mass of the water in step (2) are at a ratio of 1:(8-12).

14. The Fuzi decoction granule according to claim 13, wherein the immersion in step (2) is conducted for 25 min to 35 min.

15. The Fuzi decoction granule according to claim 10, wherein the paste has a relative density of 1.02 to 1.12.

16. The Fuzi decoction granule according to claim 10, wherein the paste and the maltodextrin are at a mass ratio of 1:0.3.

17. The Fuzi decoction granule according to claim 10, wherein the spray-drying is conducted at 165° C. to 190° C.

18. The Fuzi decoction granule according to claim 10, wherein the toxicity-removed and medicinal effect-retained Fuzi decoction granule has a particle size of 10 mesh to 16 mesh.
